(19) United States
(12) Patent Application Publication
(10) Pub. No.: US 2026/0248555 A1
(43) Pub. Date: Aug. 27, 2026

(54) CATHETER FOR FORMING A FISTULA

(71) Applicant: CLEARSTREAM TECHNOLOGIES LIMITED, Enniscorthy, County Wexford (IE)

(72) Inventors: John O'Shea, Enniscorthy, County Wexford (IE); Jakob Wells, Enniscorthy, County Wexford (IE); Michael Whelan, Enniscorthy, County Wexford (IE)

(21) Appl. No.: 18/871,154

(22) PCT Filed: Jun. 2, 2022
(86) PCT No.: PCT/EP2022/065044

§ 371 Date: Dec. 2, 2024

(51) Int. Cl. A61B 18/14; A61B 18/00

(52) U.S. Cl. CPC A61B 18/1492; A61B 2018/00214; A61B 2018/00345; A61B 2018/144

(57) ABSTRACT

A catheter for forming a fistula between two vessels. The catheter comprises a housing, an electrode disposed at least partially within the housing. The electrode comprises a distal portion, a proximal portion and an intermediate portion therebetween for contacting a vessel wall and forming the fistula. The catheter further comprises a valve destruction wire disposed at least partially within the housing for contacting and destroying a venous valve.

### TECHNICAL FIELD

The present disclosure relates to a catheter for forming a fistula between two blood vessels, a system of forming a fistula between two vessels and a method of forming a fistula using a catheter system.

### BACKGROUND

Peripheral arterial disease (PAD) can result from the occlusion of arteries in the legs and lower extremities such as the feet. Typically, such occlusion is brought about by atherosclerosis, whereby calcified plaque deposited on the walls of an arterial vessel causes narrowing and blockage of the vessel lumen. Severe cases of arterial blockage in the lower extremities can lead to critical limb ischemia (CLI), a chronic condition characterised by severe pain and slow healing of wounds in the affected extremities due to poor circulation of blood. Left untreated, the patient may suffer loss of limbs because of the need to undergo amputation.

Treatment of such diseased arteries may include angioplasty or atherectomy. However, in some circumstances, these treatments are unsuitable, and an alternative solution is necessary. One such alternative solution is deep vein arterialisation (DVA), whereby blood flow is routed from the diseased artery to a nearby deep vein in order to supply the extremity with blood. Another possible treatment is an endovascular bypass procedure, whereby blood flow is routed out of the artery and back into the artery by a conduit that circumvents the blockage. That conduit may, for example, be a stent graft placed within an adjacent vein.

The process of routing the blood from the artery to an adjacent vein involves the formation of a fistula, which is a passageway connecting the two vessels.

Further, in order for a deep vein arterialization procedure or an endovascular bypass procedure to be effective, the venous valves that normally hinder retrograde blood flow must be made incompetent.

It is known in the art to perform DVA or endovascular bypass procedures using a catheter for forming a fistula and a separate catheter for valve destruction. However, this approach may be limited, for example, by the complexity of the procedure which necessitates multiple steps involving multiple catheters.

In view of the above, there is a need for an improved catheter which can effectively form a fistula between two vessels and destroy venous valves.

There is further a need in the art for a new catheter which reduces the treatment time for a DVA or endovascular bypass procedure and makes the procedures simpler by eliminating the need for separate catheters for forming a fistula and destroying venous valves and reducing the number of steps in the procedure.

### SUMMARY

In a first aspect of the present disclosure, there is provided a catheter for forming a fistula between two vessels. The catheter comprises a housing, and an electrode disposed at least partially within the housing. The electrode comprises a distal portion, a proximal portion and an intermediate portion therebetween for contacting a vessel wall and forming a fistula. The catheter further comprises a valve destruction wire disposed at least partially within the housing for contacting and destroying a venous valve.

In some embodiments this may result in an improved catheter which can form a fistula and destroy a venous valve to facilitate effective deep vein arterialization or endovascular bypass procedure.

In some embodiments, this may reduce the treatment time for a DVA or endovascular bypass procedure and makes the procedures simpler by eliminating the need for separate catheters for forming a fistula and destroying venous valves and reducing the number of steps in the procedure

Throughout this disclosure, the term ‘fistula’ is used to denote a connection or passageway.

The valve destruction wire may be a ribbon wire.

In some embodiments result in better valve destruction.

The valve destruction wire may be a round wire.

In some embodiments this may result in better valve destruction.

The valve destruction wire may have a convex shape.

In some embodiments, this may result in better valve destruction while reducing or preventing damage to the vessel wall.

The valve destruction wire may have at least one serrated or barbed edge.

In some embodiments, this may result in better valve destruction.

The serrated or barbed edge may be disposed on a lateral side of the valve destruction wire.

In some embodiments, this may result in better valve destruction while reducing or preventing damage to the vessel wall.

Throughout this disclosure, the term ‘lateral side’ of the valve destruction wire is used to denote the sides of the valve destruction wire which are perpendicular to the radial outer side of the valve destruction wire that faces the vessel wall.

The valve destruction wire may have two serrated or barbed edges.

In some embodiments, this may result in better valve destruction.

The serrated or barbed edges may be disposed on opposite sides of the valve destruction wire.

In some embodiments, this may result in better valve destruction.

The serrated or barbed edges may be disposed on the lateral sides of the valve destruction wire.

In some embodiments, this may result in better valve destruction while reducing or preventing damage to the vessel wall.

The valve destruction wire may have an abrasive surface.

In some embodiments, this may result in better valve destruction.

Throughout this disclosure, the term ‘abrasive surface’ is used to denote a roughened surface that can scrape away the tissue of a venous valve through friction.

The valve destruction wire may have a radially expanded configuration and a radially contracted configuration.

In some embodiments, this may allow the profile of the catheter to be reduced for easier movement through a vessel.

In some embodiments, this may allow the valve destruction wire to be expanded and result in better valve destruction.

Throughout this description, the ‘radially expanded configuration’ of an element refers to a configuration where the element extends radially further from the housing than in the ‘radially contracted configuration’.

The valve destruction wire may comprise a distal end which is fixed within the housing and a proximal end which is moveable to move the valve destruction wire between the radially expanded configuration and the radially contracted configuration.

In some embodiments, this may allow the valve destruction wire to more easily move between the radially contracted configuration and the radially expanded configuration.

In at least the radially expanded position, the valve destruction wire may extend out of the housing.

In some embodiments, this may allow better separation of the direction of the electrode and the valve destruction wire.

The valve destruction wire may be disposed at the same longitudinal position as the electrode.

Throughout this description, the ‘same longitudinal position’ of the valve destruction wire as the electrode refers to a configuration in which there is at least partial overlap in the longitudinal position of the electrode and valve destruction wire.

In some embodiments, this may allow the electrode to be used in conjunction with the valve destruction wire for more efficient destruction of the venous valve.

The valve destruction wire may be disposed on the opposite side of the housing to the electrode.

In some embodiments, this may allow the valve destruction wire to help stabilize the electrode during the fistula formation process.

In some embodiments, this may result in better valve destruction.

The valve destruction wire may be a valve destruction electrode suitable for carrying an electric current.

In some embodiments this may result in better valve destruction.

The electrode may have a radially expanded configuration and a radially contracted configuration.

In some embodiments, this may allow the profile of the catheter to be reduced for easier movement through a vessel.

In some embodiments, this may allow the electrode to be expanded and result in better fistula formation.

The electrode may extend out of the housing in at least the radially expanded configuration.

The proximal end of the electrode may be longitudinally moveable to move the electrode between the radially expanded configuration and the radially contracted configuration.

The distal end of the electrode may be fixed within the housing.

In some embodiments, this may allow the electrode to more easily move between the radially contracted configuration and the radially expanded configuration.

The electrode may be a ribbon wire.

In some embodiments, this may result in better fistula formation.

The electrode may be a leaf spring.

In some embodiments, this may allow the electrode to be bent and flexed without breaking.

In some embodiments, this may allow the electrode to more easily move between the radially contracted configuration and the radially expanded configuration.

Throughout this disclosure, the term ‘leaf spring’ is used to refer to a flexible curved strip of material which can be bent but will regain its original shape when released.

The electrode may have a convex shape.

In some embodiments, this may result in better fistula formation.

The housing may be at least partly made from a ceramic material.

In some embodiments, this may allow the housing to better withstand the heat and plasma generated by the electrode.

The catheter may further comprise a ceramic spacer positioned between the electrode and the valve destruction wire.

In some embodiments, this may protect the valve destruction wire from the heat and plasma generated by the electrode during the fistula formation process.

In a second aspect of the present disclosure, there is provided a system for forming a fistula between two vessels. The system comprises a first catheter according to any of the preceding clauses.

The system may further comprise a second catheter comprising a second housing and a backstop for the electrode.

The backstop may be a recessed backstop which has a portion shaped complementary to the electrode.

In some embodiments, this may allow the electrode to better engage with the backstop and result in better fistula formation.

The backstop may have a concave portion.

The first catheter and the second catheter may each comprise one or more magnets positioned to align the electrode with the backstop.

In some embodiments, this may provide a simple way to allow exact alignment of the electrode and the backstop.

The system may further comprise a radiofrequency generator for supplying radiofrequency power to the electrode.

The radiofrequency generator may be configured to supply radiofrequency power to the valve destruction wire.

In some embodiments, this may result in a more efficient use of the radiofrequency generator.

The system may further comprise a handle disposed at the proximal end of the first catheter.

The handle may comprise an electrode expansion mechanism.

The electrode expansion mechanism may comprise a slider for moving a proximal end of the electrode to move the electrode between the radially expanded configuration and the radially contracted configuration.

In some embodiments, this may provide a simpler way to independently control movement of the electrode between the radially expanded and radially contracted configurations.

The handle may comprise a valve destruction wire expansion mechanism.

The valve destruction wire expansion mechanism may comprise a slider for moving a proximal end of the valve destruction wire to move the valve destruction wire between the radially expanded configuration and the radially contracted configuration.

In some embodiments, this may provide a simpler way to independently control movement of the valve destruction wire between the radially expanded and radially contracted configurations.

In a third aspect of the present disclosure, there is provided a method for forming a fistula using a catheter having a housing with an electrode and a valve destruction wire at least partially disposed within the housing. The method comprises inserting the catheter into a vein through an access site, advancing the catheter to a treatment site where the fistula is to be formed, moving the electrode from a radially contracted position to a radially expanded position and applying RF energy to the electrode to form the fistula. The method further comprises moving the valve destruction wire from the radially contracted position to a radially expanded position, moving the valve destruction wire to a valve in the vein which is to be destroyed and rotating the catheter while moving it longitudinally back and forth to destroy the valve.

In a fourth aspect of the present disclosure, there is provided a method of forming a catheter for forming a fistula between two vessels. The method comprises forming a housing; disposing an electrode at least partially within the housing, the electrode comprising a distal portion, a proximal portion and an intermediate portion therebetween for contacting a vessel wall and forming the fistula; and disposing a valve destruction wire at least partially within the housing for contacting and destroying a venous valve.

The method may further comprise forming at least one serrated or barbed edge on the valve destruction wire.

The at least one serrated or barbed edge may be formed on a lateral side of the valve destruction wire.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows a catheter system for forming a fistula according to the present disclosure.

FIG. 2A shows a perspective view of a valve destruction wire having a ribbon shape and serrated edges.

FIG. 2B shows a perspective view of an alternative embodiment of a valve destruction wire having a ribbon shape and barbed edges.

FIG. 2C shows a perspective view of an alternative embodiment of a valve destruction wire having a round shape and serrations.

FIG. 2D shows a perspective view of an alternative embodiment of a valve destruction wire having a round shape and barbs.

FIG. 3A shows a cross-sectional side view of the catheter system of FIG. 1 being deployed in a blood vessel system prior to forming a fistula.

FIG. 3B shows a cross-sectional side view of the catheter system of FIG. 1 disposed at the treatment site for forming a fistula.

FIG. 3C shows a cross-sectional side view of the catheter system of FIG. 1, during the valve destruction process.

FIG. 3D shows a cross-sectional side view of a blood vessel system following completion of fistula formation and venous valve destruction.

### DETAILED DESCRIPTION

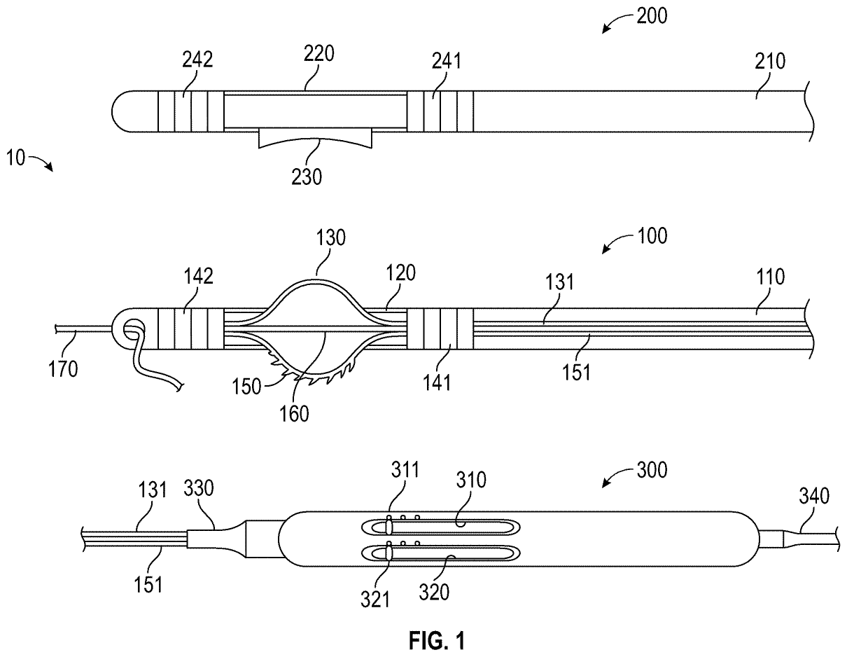

FIG. 1 shows a catheter system 10 for forming a fistula. The system comprises a first catheter 100 and a second catheter 200 which can be used together to form a fistula between two vessels. The system may further comprise a handle 300 disposed at the proximal end of the first catheter 100.

The first catheter 100 comprises a shaft 110 and a housing 120 disposed at the distal end of the shaft 110. An electrode 130 is at least partially disposed in the housing 120 and may extend out of the housing 120 through a first opening in the housing 120. The electrode 130 may have a convex shape. The electrode 130 may further have a radially contracted configuration and a radially expanded configuration. FIG. 1 shows the electrode 130 in the radially expanded configuration, where the electrode 130 may extend radially further from the housing than in the radially contracted configuration. In the radially contracted configuration, the electrode 130 may be fully disposed within the housing 120 or it may extend from the housing 120 by a small distance. A first connecting element 131 may be connected to a proximal end of the electrode 130 and may extend along the length of the shaft 110 of the first catheter 100. The first connecting element 131 may be connected to a source of RF energy via the handle 300. For example, a proximal portion 340 of the handle 300 may be connected to a source of RF energy such as an ESU pencil, for example. The first connecting element 131 may be conductive to allow RF energy to be supplied to the electrode 130 via the first connecting element 131.

The catheter 100 further comprises a valve destruction wire 150 which is at least partially disposed within the housing 120 and may extend out of the housing through a second opening in the housing 120. The valve destruction wire 150 may have a convex shape. The valve destruction wire 150 may also have a radially contracted configuration and a radially expanded configuration. FIG. 1 shows the valve destruction wire 150 in the radially expanded configuration, where the valve destruction wire 150 may extend radially further from the housing 120 than in the radially contracted configuration. In the radially contracted configuration, the valve destruction wire 150 may be fully disposed within the housing 120 or it may extend from the housing 120 by a small distance. A second connecting element 151 may be connected to a proximal end of the valve destruction wire 150 and may extend along the length of the shaft 110 of the first catheter 100. The second connecting element 151 may also be connected to a source of RF energy via the handle 300. In some embodiments, the second connecting element 151 may also be conductive to allow RF energy to be supplied to the valve destruction wire 150 via the second connecting element 151.

As shown in FIG. 1, the electrode 130 and the valve destruction wire 150 may be disposed on opposite sides of the housing 120 and extend in opposite radial directions. Further, the electrode 130 and the valve destruction wire 150 may be disposed at the same longitudinal position.

The catheter 100 may also comprise a ceramic spacer 160 positioned between the electrode 130 and the valve destruction wire 150 within the housing 120 for electrically isolating the electrode 130 from the valve destruction wire 150. The catheter 100 may further comprise a proximal set of magnets 141 and a distal set of magnets 142 which are disposed proximally and distally of the housing 120, respectively.

The electrode 130 may be in the form of a ribbon wire and may be made from a number of suitable materials, such as one or more refractory metals. For example, the electrode 130 may comprise tungsten, molybdenum, niobium, tantalum, rhenium, or combinations and alloys thereof. The valve destruction wire 150 may be made from the same materials as the electrode 130 or it may be made, for example, from nitinol. The housing 120 may be made from a non-conductive ceramic material which can withstand the heat and plasma generated by the electrode 130 and/or the valve destruction wire 150.

The second catheter 200 also comprises a catheter shaft 210 and a second housing 220 having a backstop 230 disposed at the distal end of the shaft 210. The backstop 230 may have a concave portion which is shaped complimentary to the convex shape of the electrode 130. The second catheter 200 and backstop 230 may also be made from a ceramic material to withstand the heat and plasma generated by the electrode 130. The second catheter 200 may also comprise a proximal set of magnets 241, disposed proximally of the housing 220, and a distal set of magnets 242, disposed distally of the housing 220.

The handle 300 may have an electrode expansion mechanism 310, which comprises a moveable electrode slider 311 that is connected to the proximal end of the first connecting element 131. The distal end of the first connecting element 131 is connected to the proximal end of the electrode 130 and the distal end of the electrode 130 is fixed to the housing 120. In order to move the electrode 130 from the radially contracted configuration to the radially expanded configuration, a user can push the electrode slider 311 in a distal direction. Contrastingly, in order move the electrode 130 from the radially expanded configuration to the radially contracted configuration, user can push the electrode slider 311 in a proximal direction.

The handle 300 may further include a valve destruction wire expansion mechanism 320, which comprises a moveable valve destruction wire slider 321 that is connected to the proximal end of the second connecting element 151. The distal end of the second connecting element 151 is connected to the proximal end of the valve destruction wire 150. The distal portion of the valve destruction wire 150 is fixed to the housing 120. In order to move the valve destruction wire from the radially contracted configuration to the radially expanded configuration, a user can push the valve destruction wire slider 321 in a distal direction. Contrastingly, in order to move the valve destruction wire 150 from the radially expanded configuration to the radially contracted configuration, a user can push the valve destruction wire slider 321 in a proximal direction. The handle 300 thereby allows the electrode 130 and the valve destruction wire 150 to be independently moved between their respective radially contracted configuration and radially expanded configuration.

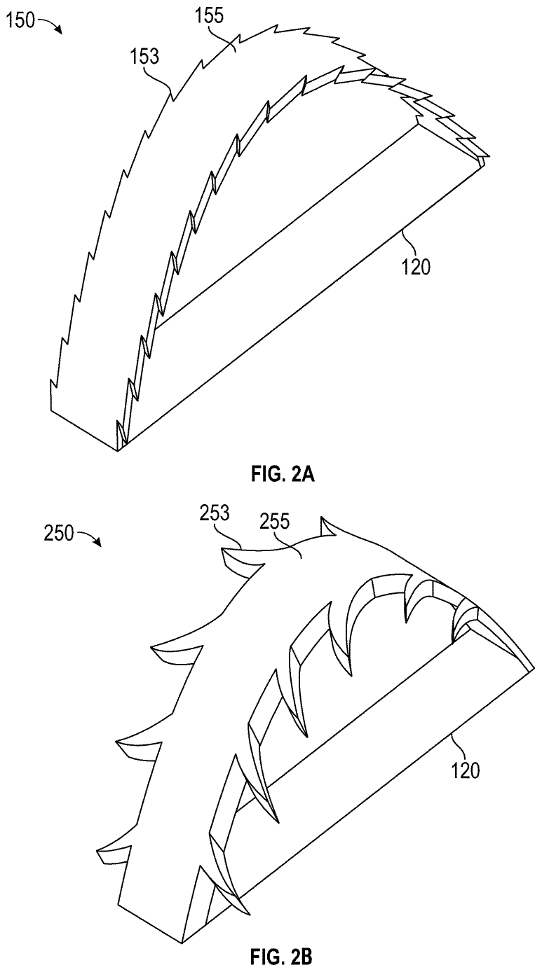

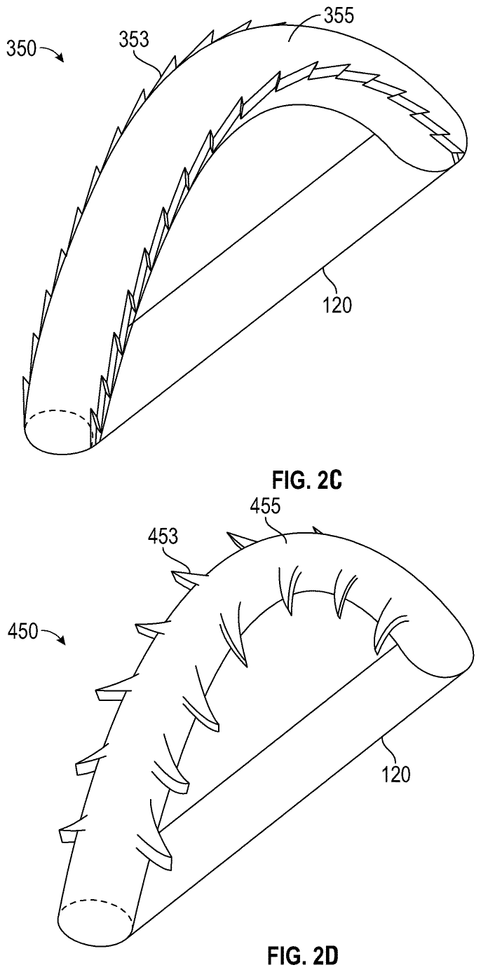

FIG. 2A shows a perspective view of a valve destruction wire 150 having a ribbon shape and serrated edges 153. In some embodiments, the valve destruction wire 150 has at least one serrated edge. The serrated edge 153 comprises a plurality of teeth which can better destroy the tissue of a valve, for example by ripping, scraping or cutting, when moved into contact with the valve. In FIG. 2A, two serrated edges 153 are disposed on opposite lateral sides of the valve destruction wire 150. These lateral sides of the valve destruction wire 150 come into contact with the valve when the valve destruction wire 150 is rotated around the inner circumference of a vein. The radial outer surface 155 of the valve destruction wire 150 is non-serrated or atraumatic. By positioning the serrated edges 153 on the lateral sides of the valve destruction wire 150 this results in minimal contact of the serrated edges 153 with the vessel wall. Therefore, valve destruction wire 150 shown in FIG. 2A results in effective valve destruction whilst also preventing or reducing damage to the vessel wall. The convex shape of the valve destruction wire 150 is also advantageous for reducing damage to the vessel wall as it allows the valve to be more accurately targeted by the valve destruction wire 150.

Those skilled in the art will appreciate that many suitable surface and cross-sectional shapes of the valve destruction wire 150 can be used. For example, FIG. 2B shows a perspective view of an alternative embodiment of a valve destruction wire 250. Throughout this disclosure, the same reference numerals will be used to denote features which are identical across different embodiments. The valve destruction wire 250 also has a ribbon shape but has barbed edges 253. The barbed edges 253 may similarly be positioned on the lateral sides of the valve destruction wire 250. The barbed edges 253 may comprise a plurality of barbs to better destroy the tissue of a valve, for example by ripping, scraping or cutting, when moved into contact with the valve. The radial outer surface 255 of the valve destruction wire 250 is non-barbed or atraumatic. FIG. 2C shows a perspective view of an alternative embodiment of a valve destruction wire 350 with a round cross section and with serrated edges 353 on its surface. These serrated edges 353 are similar to the serrated edges 153 of FIG. 2A and also comprise a plurality of teeth for better destroying the valve tissue. The serrated edges 353 are also disposed on two lateral sides of the valve destruction wire 350. The radial outer surface 355 of the valve destruction wire 350 is non-serrated or atraumatic. FIG. 2D shows a perspective view of an alternative embodiment of a valve destruction wire 450 with a round cross section and two barbed edges 453 disposed on lateral sides of the valve destruction wire 450. The barbed edges 453 are similar to the barbed edges 253 of FIG. 2B and comprises a plurality of barbs for better destroying the valve tissue. The radial outer surface 455 of the valve destruction wire 450 is non-barbed or atraumatic.

In some embodiments, the valve destruction wire 150 may have an abrasive surface for scraping and grinding the valve tissue and thereby better destroying the valve. Such an abrasive surface could be made by electropolishing, chemical etching or any suitable treatment of the valve destruction wire 150. The abrasive surfaces may only be provided on the lateral sides of the valve destruction wire 150 while the non-lateral sides are atraumatic to reduce the risk of vessel trauma.

Each of the valve destruction wires 150, 250, 350 and 450 can be used as part of first catheter 100.

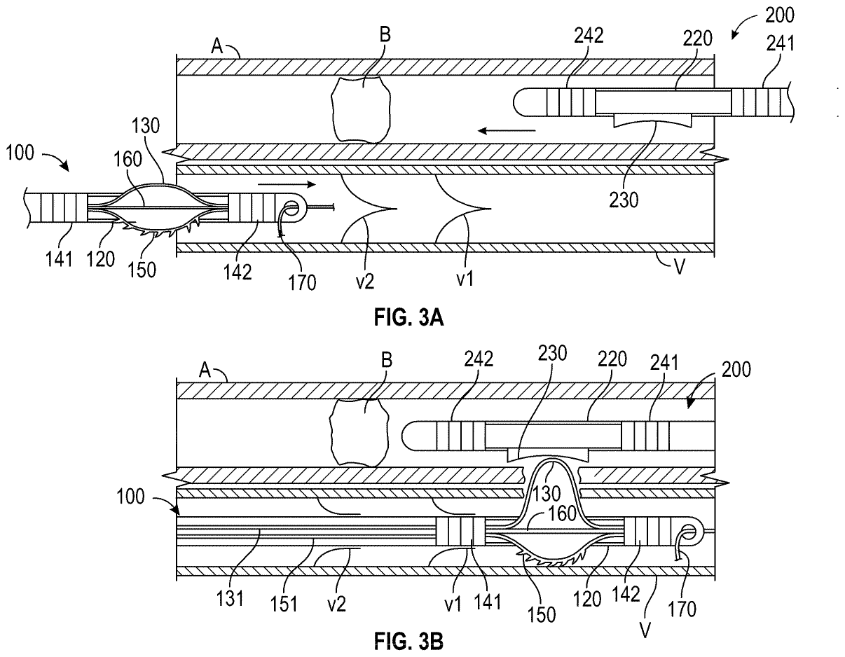

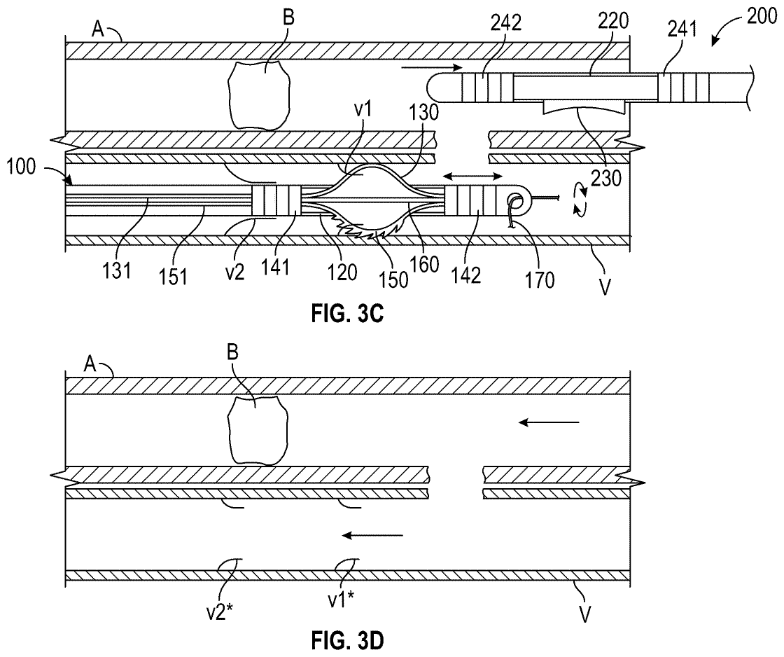

FIGS. 3A-D illustrate a method of forming a fistula between an artery A and vein V and destruction of valves in the vein V in order to circumvent a blockage B in the artery A using the catheter system 10.

As shown in FIG. 3A, firstly, the first catheter 100 is introduced into a vein V through an access site and the second catheter 200 is introduced into an artery A through a second access site.

The first catheter 100 is then advanced through the vein V toward the treatment site where the fistula is to be formed. The first catheter 100 may be introduced and advanced to the treatment site along a guidewire 170. Similarly, the second catheter 200 is advanced through the artery A toward the treatment site where the fistula is to be formed. The second catheter 200 may also be advanced to the site where the fistula is to be formed along a guidewire (not shown). The first catheter 100 and/or the second catheter 200 may be advanced to the treatment site inside a sheath. The catheter 100 and the second catheter 200 may be advanced to the treatment site from opposite directions, as shown in FIG. 3A, or the same direction.

The electrode 130 and the valve destruction wire 150 are shown in FIG. 3A in the radially contracted configuration. This allows the first catheter 100 to be more easily introduced and advanced through the vein V. If a sheath is used to introduce the catheter 100 into a vessel, the radially contracted configuration allows the first catheter 100 to more easily fit into the sheath.

Once the first catheter 100 and the second catheter 200 are positioned at the treatment site, as shown in FIG. 3B, the proximal set of magnets 141 of the catheter will be attracted to the distal set of magnets 242 of the second catheter and align themselves with each other. Similarly, the distal set of magnets 142 of the catheter 100 will be attracted to the proximal set of magnets 241 of the second catheter 200 and these sets of magnets will align with each other. This will result in the electrode 130 becoming aligned with the backstop 230. The sets of magnets may also have the effect of pulling the artery A and vein V closer together.

The electrode 130 may then be moved by a user from the radially contracted configuration to the radially expanded configuration, as shown in FIG. 3B. This may be done by pushing the slider 311 of the electrode expansion mechanism 310 and the slider 321 of the valve destruction wire expansion mechanism 320 in a distal direction (FIG. 1). In the radially expanded configuration, the electrode 130 has an increased electrode height, which allows the electrode 130 to more effectively cut through the vessel walls to form a fistula.

A radiofrequency (RF) current may then be supplied to the electrode 130 which causes the electrode 130 to heat up and generate a plasma. The plasma causes rapid dissociation of the molecular bonds in the organic compounds and allows the electrode 130 to cut through the venous and arterial vessel walls until it hits the backstop 230 to form a fistula.

The ceramic spacer 160 positioned between the electrode 130 and the valve destruction wire 150 can protect the valve destruction wire 150 from the heat and plasma generated by the electrode during the fistula forming process.

During the fistula formation process, the valve destruction wire 150 may also be moved into the radially expanded configuration, using the valve destruction wire expansion mechanism 320. Since the valve destruction wire 150 is disposed on the opposite side of the housing 120 to the electrode 130, as shown in FIG. 3B, the valve destruction wire 150 may come into contact with the opposite side of the venous wall. This helps with stabilising of the electrode 130 during the fistula formation process and also helps to push the electrode 130 against the vessel wall to more effectively form the fistula.

Once the fistula is formed, the first catheter 100 may be retracted from the site of the fistula to the location of a first venous valve v1. During this re-positioning of the first catheter 100, the electrode 130 and the valve destruction wire 150 can be in their radially contracted configurations so that the first catheter 100 is more easily moved to the site of the valve v1.

As shown in FIG. 3C, when the location of the first venous valve v1 is reached, the valve destruction wire 150 may be deployed in its radially expanded configuration. The first catheter 100 may then be rotated. Rotation of the first catheter 100 causes the lateral surfaces of the valve destruction wire 150, which may comprise serrations 153, 353 barbs 253, 453 or other abrasive surfaces (see FIGS. 2A-2D), to contact and damage the valve tissue of venous valve v1, for example by ripping, scraping, cutting. The rotational movement of the catheter 100 during the valve destruction process may also be supplemented by short longitudinal movements of the catheter 100 to improve the effectiveness of valve destruction. The first venous valve v1 is thereby rendered incompetent such that it can no longer prevent retrograde blood flow in the vein V. This process of valve destruction can be repeated to destroy a second venous valve v2 and any number of valves as necessary for the treatment procedure.

In some embodiments, as shown in FIG. 3C, the electrode 130 may also be deployed in the radially expanded configuration during the process of valve destruction, i.e. it may be used in conjunction with the valve destruction wire 150 to destroy the valve v1. For example, the electrode 130 in the radially expanded configuration can scrape or abrade the valve tissue to help make the valve v1 incompetent. By having the electrode 130 and the valve destruction wire 150 disposed at the same longitudinal position, the electrode 130 may also help to apply pressure to the valve destruction wire 150 against the valve tissue which may result in more effective valve destruction.

In some embodiments, the valve destruction wire 150 may carry an electric current during the valve destruction process. This may result in more effective destruction of the valve v1, for example by ablation of the valve tissue through heating or plasma vaporisation.

FIG. 3D shows a cross-sectional side view of a blood vessel system following completion of the fistula formation and venous valve destruction processes according to FIGS. 3B-3C.

The arrows in FIG. 3D depict the flow of blood from the artery A through the fistula and into the vein V, and then through the incompetent valves v1* and v2* in the distal direction. In this case, the vein V is successfully arterialised after deployment of the catheter system 10. The blood flow that is initially blocked by the blockage B in the artery A is re-routed to the vein V and can effectively flow in a retrograde direction in the vein V without hindrance by the venous valves.

When performing a deep vein arterialization (DVA) procedure, a stent may be placed within the fistula to stabilise the fistula. When performing an endovascular bypass procedure, a second fistula may be formed distally of the blockage B in a similar manner as explained with respect to FIG. 3B above. A stent graft may then be placed through the first and second fistulas via the vein V, such that the blood flow can circumvent the blockage B.

Various modifications will be apparent to those skilled in the art.

The valve destruction wire 150 is not limited to a ribbon wire or round wire, but may be any other type of suitable wire, for example an oval wire.

The valve destruction wire 150 is not limited to a convex shape, but may be any other type of suitable shape, for example a rectangular shape, trapezoidal shape or triangular shape.

The valve destruction wire 150 may not have any serrations or barbs or abrasive surfaces. Alternatively, only one side of the valve destruction wire 150 may have serrations or barbs or an abrasive surface.

The valve destruction wire 150 may have opposing lateral sides which may have different types of surfaces. For example, one side may have a serrated edge whereas the other side may have a barbed edge. Alternatively, each lateral side may have a combination of serrations, bards or abrasive surfaces.

The valve destruction wire 150 is not limited to one wire, but the first catheter 100 may comprise a plurality of valve destruction wires 150. For example, the first catheter 100 may comprise two valve destruction wires disposed at a 120 degree angle to each other.

The electrode 130 and valve destruction wire 150 are not limited to being positioned on opposite sides of the housing, but may be positioned at different radial angles a to each other.

The electrode 130 is not limited to a ribbon wire, but may be any other type of suitable wire, for example, a cylindrical wire or oval wire.

The electrode 130 is not limited to a convex shape, but may be any other type of suitable shape, for example a rectangular shape, trapezoidal shape or triangular shape.

The spacer 160 positioned between the electrode 130 and the valve destruction wire 150 is not limited to a ceramic material, but may be made from any suitable material which can protect the valve destruction wire 150 from the heat and plasma generated by the electrode 130.

The backstop 230 of the second catheter 200 is not limited to a concave shape but may also be any suitable shape. For example, the backstop 230 may be recessed or protruding and could have a concave, convex or rectangular shape.

The housing 120 of the catheter is not limited to a ceramic material and may be made from any suitable material which can withstand the heat and plasma generated by the electrode 130. The electrode expansion mechanism 310 is not limited to a slider, but may comprise any suitable mechanism which can move the electrode between the radially expanded configuration and the radially contracted configuration.

The valve destruction wire expansion mechanism 320 is not limited to a slider, but may comprise any suitable mechanism which can move the valve destruction wire between the radially expanded configuration and the radially contracted configuration.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A catheter for forming a fistula between two vessels, comprising:
a housing;
an electrode disposed at least partially within the housing, the electrode comprising a distal portion, a proximal portion and an intermediate portion therebetween for contacting a vessel wall and forming the fistula;
a valve destruction wire disposed at least partially within the housing for contacting and destroying a venous valve.

2. The catheter of claim 1, wherein the valve destruction wire is a ribbon wire or a round wire.

3. (canceled)

4. The catheter of claim 1, wherein the valve destruction wire has a convex shape.

5. The catheter of claim 1, wherein valve destruction wire has at least one serrated or barbed edge.

6. (canceled)

7. The catheter of claim 1, wherein the valve destruction wire has two serrated or barbed edges.

8. (canceled)

9. The catheter of claim 7, wherein the serrated or barbed edges are disposed on the lateral sides of the valve destruction wire.

10. The catheter of claim 1, wherein the valve destruction wire has an abrasive surface.

11. The catheter of claim 1, wherein the valve destruction wire has a radially expanded configuration and a radially contracted configuration.

12. The catheter of claim 1, wherein the valve destruction wire comprises a distal end which is fixed within the housing and a proximal end which is moveable to move the valve destruction wire between the radially expanded configuration and the radially contracted configuration.

13. The catheter of claim 1, wherein, in at least the radially expanded position, the valve destruction wire extends out of the housing.

14. The catheter of claim 1, wherein the valve destruction wire is disposed at the same longitudinal position as the electrode.

15. The catheter of claim 1, wherein the valve destruction wire is disposed on the opposite side of the housing to the electrode.

16-22. (canceled)

23. The catheter of claim 1, wherein the housing is at least partly made from a ceramic material.

24. The catheter of claim 1, further comprising a ceramic spacer positioned between the electrode and the valve destruction wire.

25. A system for forming a fistula between two vessels comprising:
a first catheter according to claim 1 and further comprising a second catheter comprising a second housing and a backstop for the electrod.

26. (canceled)

27. The system of claim 25, wherein the backstop is a recessed backstop which has a portion shaped complementary to the electrode.

28. The system of claim 25, wherein the backstop has a concave portion.

29. The system of claim 25, wherein the first catheter and the second catheter each comprise one or more magnets positioned to align the electrode with the backstop.

30-31. (canceled)

32. The system of claim 25, further comprising a handle disposed at the proximal end of the first catheter, the handle comprises an electrode expansion mechanism, wherein the electrode expansion mechanism comprises a slider for moving a proximal end of the electrode to move the electrode between the radially expanded configuration and the radially contracted configuration.

33-34. (canceled)

35. The system of claim 32, wherein the handle comprises a valve destruction wire expansion mechanism.

36. The system of claim 35, wherein the valve destruction wire expansion mechanism comprises a slider for moving a proximal end of the valve destruction wire to move the valve destruction wire between the radially expanded configuration and the radially contracted configuration.

37. A method of forming a fistula using a catheter having a housing with an electrode and a valve destruction wire at least partially disposed within the housing, the method comprising:
inserting the catheter into a vein through an access site;
advancing the catheter to a treatment site where the fistula is to be formed;
moving the electrode from a radially contracted position to a radially expanded position;
supplying RF energy to the electrode to form the fistula;
moving the valve destruction wire from a radially contracted position to a radially expanded position;
moving the valve destruction wire to a valve in the vein which is to be destroyed;
rotating the catheter while moving it longitudinally back and forth to destroy the valve.

38-40. (canceled)
